(19) United States
(12) Patent Application Publication
(10) Pub. No.: US 2021/0393856 A1
(43) Pub. Date: Dec. 23, 2021

(54) COMPOSITIONS AND METHODS FOR REDUCING CALCIFICATION OF HEART VALVES

(71) Applicant: Arizona Board of Regents on Behalf of the University of Arizona, Tucson, AZ (US)

(72) Inventors: Zain Khalpey, Tucson, AZ (US); Raymond Runyan, Tucson, AZ (US)

(21) Appl. No.: 17/279,152

(22) PCT Filed: Sep. 25, 2019
(86) PCT No.: PCT/US2019/052914

§ 371 Date: Mar. 24, 2021

Related U.S. Application Data
(60) Provisional application No. 62/736,262, filed on Sep. 25, 2018.

(51) Int. Cl. A61L 27/54; A61K 38/46; A61L 27/34

(52) U.S. Cl. CPC A61L 27/54; A61K 38/46; A61K 38/465; A61L 2300/254; C12Y 301/03005; A61L 2430/20; A61L 27/34

(57) ABSTRACT

Provided herein are compositions and methods for preventing calcification of replacement heart valves. In particular, provided herein are compositions and methods for increasing local production of adenosine on or in the valve.

### CROSS REFERENCE TO RELATED APPLICATION

This application claims priority to and the benefit of U.S. Provisional Application No. 62/736,262, filed Sept. 25, 2018, which is hereby incorporated by reference in its entirety.

### FIELD OF THE DISCLOSURE

Provided herein are compositions and methods for preventing calcification of replacement heart valves (e.g., xenotic or cadaveric valves). In particular, provided herein are compositions and methods for increasing local production of adenosine on or in the valve.

### BACKGROUND OF THE DISCLOSURE

Heart valve disease (also called valvular heart disease, VHD) occurs when one of the four valves of the heart is not working properly. More than five million Americans are diagnosed with heart valve disease each year (2006 numbers). The aortic and the mitral valves are the most commonly affected valves. VHD can be treated via surgery to repair or replace diseased heart valves. There are two categories of replacement valves: mechanical (non-biological materials) and tissue valves that can be inserted by open-heart surgery (Erin M. Spinner. Bridge, Frontiers of Engineering. March 2015; vol 15, issue 1). Tissue-based, or bio-prosthetic, valves are made from biological heart tissue (pig, cow or human). Aortic valves can also be replaced by transcatheter aortic valve replacement (TAVR), which is less invasive and involves delivering the replacement valve through an artery in the leg or the arm. Mechanical valves do not need to be replaced, but they require the constant use of anti-coagulants. In contrast, tissue valves do not require anti-coagulant medication but lack the durability of mechanical valves. On average, tissue valves last 10 to 20 years before requiring replacement. Mainly, calcification of the bio-prosthetic valves lowers their life expectancy, requiring additional surgery and cost to the patient (Schoen F J and Levy R J. The Annals of Thoracic Surgery. 2005; 79: 1072-80). Despite their low durability, tissue-based valves are preferred by patient to avoid life-long anti-coagulant medication.

Almost all of the current generation of biological valves have some form of anti-calcification treatment. Most of them work by preventing the binding of calcium molecules to fat molecules on the valve leaflets. Ultimately, when calcification does occur, replacement surgery is usually the only option.

Additional compositions and methods for preventing calcification of implantable valves are needed.

### SUMMARY OF THE DISCLOSURE

Experiments described herein demonstrated that extracellular adenine nucleotide metabolism can affect degeneration and calcification of aortic valve prostheses. Adenosine is a known regulator of calcification within the cardiovascular system and is produced through the degradation of ATP/ADP and AMP. Studies were performed on the activity of two ADP and AMP modifying enzymes: ecto-5′-nucleotidase (CD73) and ecto-nucleoside triphosphate diphosphohydrolase 1 (CD39). These enzymes are present on the surface of native intact valve and are affected by the various treatments that are required to sterilize and decrease immunogenicity of the tissue-based replacement valve (Kutryb-Zajac et al., Journal of Cardiovascular Translational Research. 2016; 9:119-126).

Accordingly, provided herein are compositions and methods for modifying replacement valves by coupling an enzyme that generated adenosine (e.g., CD39 or CD73) to the valve surface. The localized production of adenosine inhibits or reduces valve calcification and extends the life of the replacement valve, decreasing the likelihood of replacement surgery in the future and thus preventing costly and risky surgery.

For example, in some embodiments, provided herein is an implantable medical valve comprising one or more enzymes that generate adenosine. The present disclosure is not limited to particular enzymes. For example, in some embodiments, the enzyme is an ecto-nucleotidase or ecto-nucleosidase (e.g., a 5′ ectonucleotidase, an ecto-nucleoside triphosphate diphosphohydrolase 1, a nucleotide pyrophosphatase/phosphodiesterase (NPP)-type ecto-phosphodiesterase, or an alkaline phosphatase). In some embodiments, the 5′ ectonucleotidase is CD73. In some embodiments, the ecto-nucleoside triphosphate diphosphohydrolase 1 is CD39. In some embodiments, a combination of CD39 and CD73 is used.

In some embodiments, the enzyme is modified to prevent proteolysis of the enzyme (e.g., to improve half-life in vivo). The present disclosure is not limited to particular modifications. Examples include, but are not limited to, removal of proteolytic target sites and truncation of the enzyme. In some embodiments, truncation retains the portion of said enzyme that produces adenosine. In some embodiments, the enzyme is linked to a targeting agent that binds to the medical valve (e.g., via a cell surface marker or binding target such as a hapten). In some embodiments, the targeting agent is an antibody.

The present disclosure is suitable for any number of different types of valves. In one exemplary embodiment, the valve is a heart valve. For example, in some embodiments, the heart valve is a tissue (e.g., cadaveric valve), a xenobiotic valve, a homograft, an autograft, or a synthetic valve. In some embodiments, the valve is a valve for use in Transcatheter Aortic Valve Replacement (TAVR).

The present disclosure is not limited to a particular configuration or method of providing the enzyme to the valve. In some embodiments, the enzyme is present on the surface of the valve or integrated into the valve. For example, in some embodiments, the enzyme is cross-linked to the valve. In some embodiments, the enzyme is secreted by a cell that expresses the enzyme.

Further embodiments provide a system, comprising: a) an implantable medical valve; and b) a source of an enzyme that generates adenosine. The present disclosure is not limited by the method of providing the enzyme. In some embodiments, the enzyme is secreted by a cell that expresses said enzyme or is in solution.

Additional embodiments provide a method of preventing calcification of an implantable medical valve, comprising: providing a valve or system as described herein to a subject in need thereof.

Further embodiments provide the use of a valve or system as described herein to prevent calcification of an implantable medical valve.

In some embodiments, the enzyme provided to the valve prior to or during implantation of the valve in the subject. In some embodiments, the providing is repeated (e.g., by administering an enzyme with a targeting agent to the subject one or more times as needed).

Additional embodiments are described herein.

### BRIEF DESCRIPTION OF THE DRAWINGS

FIG. 1 shows valve calcification in vitro. Left panel shows a Day 0 (6 hr) culture. Right panel shows calcification in vitro by 7 days.

FIG. 2 shows Runx2 isoform expression in calcifying valves.

FIG. 3 shows micromass culture of Mouse AV Mesenchyme Cell Line compared to L-Cell line.

FIG. 4 shows ecto-nucleases on the cell surface.

FIG. 5 shows activity of 1. eNTPD, 2. e5NT, 3 eADA on the fibrosa surface of calcified aortic valve (n=101) vs. non-calcified aortic valve (n=19). Data are shown as mean±SD, *p<0.05, **p<0.01, **p<0.001.

FIG. 6 shows that the CD73−/− mouse demonstrates aortic valve calcification. *P<0.05 by Two-way ANOVA.

FIG. 7 shows that Runx2 expression is up-regulated during human aortic valve calcification.

FIG. 8 shows that adenosine receptor agonists upregulate osteogenic markers in mouse AVM cell cultures.

FIG. 9 shows that calcifying AVM cells show reduced Runx2-II and Mgp in the presence of AMP.

FIG. 10 shows histological demonstration of CD73 activity.

FIG. 11 shows adenosine effects on Runx2 expression. Expression was normalized to GAPDH. Average of 3 independent repeats with SEM.

FIG. 12 shows qPCR measurement of Runx2-II. Runx2 expression increases in calcifying AVM cells with time in culture. Cells treated with either 2′-MeCCPA (A1 agonist) or PSB0777 (A2a agonist) up to 5 days.

FIG. 13 shows qPCR measurement of osteogenic marker expression by AVM cell cultures.

FIG. 14 shows adenosine receptor agonist treatment. A1 (Adora1) transcript expression normalized by GAPDH in human VICs treated with 0.1 μM of 2′-MeCCPA (A1 agonist) and 1 μM of PSB0777 (A2a agonist) up to 5 days.

### DEFINITIONS

As used herein, the term “subject” refers to individuals (e.g., human, animal, or other organism) to be treated by the methods or compositions of the present invention. Subjects include, but are not limited to, mammals (e.g., murines, simians, equines, bovines, porcines, canines, felines, and the like), and most preferably includes humans.

As used herein, the term “administration” refers to the act of giving a drug, prodrug, or other agent, or therapeutic treatment (e.g., a heart valve comprising an intervention described herein) to a physiological system (e.g., a subject or in vivo, in vitro, or ex vivo cells, tissues, and organs).

As used herein, the term “non-human animals” refers to all non-human animals including, but are not limited to, vertebrates such as rodents, non-human primates, ovines, bovines, ruminants, lagomorphs, porcines, caprines, equines, canines, felines, ayes, etc.

As used herein, the term “sample” is used in its broadest sense. In one sense, it is meant to include a specimen or culture obtained from any source, as well as biological and environmental samples. Biological samples may be obtained from animals (including humans) and encompass fluids, solids, tissues, and gases. Biological samples include blood products, such as plasma, serum and the like. Such examples are not however to be construed as limiting the sample types applicable to the present invention.

### DETAILED DESCRIPTION OF THE DISCLOSURE

As described herein, provided herein are improved compositions, systems, and methods for preventing calcification of implanted valves (e.g., heart valves). Exemplary compositions and methods are described herein.

The present disclosure provides systems for increasing local concentrations of adenosine in or on implantable valves. In some embodiments, the present disclosure provides enzymatic systems for generating adenosine. In some embodiments, the present disclosure utilizes ecto-nucleotidases. Ectonucleotidases are nucleotide metabolizing enzymes that are expressed on the plasma membrane and have externally oriented active sites. These enzymes metabolize nucleotides to nucleosides. The contribution of ectonucleotidases in the modulation of purinergic signaling depends on the availability and preference of substrates and on cell and tissue distribution. Ectonucleotidases produce key molecules for purine salvage and consequent replenishment of ATP stores within multiple cell types. Dephosphorylated nucleoside derivatives interact with membrane transporters to enable intracellular uptake. Ectonucleotidases modulate P2 purinergic signaling. In addition, ectonucleotidases generate extracellular adenosine, which abrogates nucleotide-mediated effects and activates adenosine receptors, often with opposing (patho-) physiological effects.

The first step in the production of adenosine involves the conversion of ATP/ADP to AMP. It is carried out by ENTPD1, also known as CD39. The second step involves the conversion of AMP to adenosine. It is carried out by NT5E, also known as CD73.

Accordingly, in some embodiments, one or more ectonucleotidases (e.g., a 5′ ectonucleotidase, an ecto-nucleoside triphosphate diphosphohydrolase 1, a nucleotide pyrophosphatase/phosphodiesterase (NPP)-type ecto-phosphodiesterase, or an alkaline phosphatase) are used to generate adenosine. In some embodiments, the 5′ ectonucleotidase is CD73 (e.g., Homo sapiens CD73 described by GenBank accession numbers BC015940.1 and BC065937.1). In some embodiments, the ecto-nucleoside triphosphate diphosphohydrolase 1 is CD39 (e.g., Homo sapiens CD39 described by GenBank accession number S73813.1 or NM_001776.6). In some embodiments, a combination of CD39 and CD73 is used.

In some embodiments, the enzyme is modified to prevent proteolysis of the enzyme (e.g., to improve half-life in vivo). The present disclosure is not limited to particular modifications. Examples include, but are not limited to, removal of proteolytic target sites and truncation of the enzyme. In some embodiments, a version of CD73 with the final 40 nucleotides of CD73 described by GenBank BC065937.1 removed is utilized. In some embodiments, a version of CD39 described by GenBank accession number S73813.1 or NM_001776.6 with the first 110 and the last 138 nucleotides removed is utilized. In some embodiments, truncation retains the portion of said enzyme that produces adenosine. In some embodiments, the enzyme is linked to a targeting agent that binds to the medical valve (e.g., via a cell surface marker or binding target such as a hapten). In some embodiments, the targeting agent is an antibody.

The compositions and methods for increasing production of adenosine described herein find use with a variety of different implantable medical valves. While the disclosure is exemplified with heart valves, the technology is not limited to heart valves and finds use in any application where production of adenosine is desired.

In some embodiments, the compositions and methods described herein find use in preventing calcification of heart valves (e.g., replacement valves). The present disclosure is not limited to particular types of heart valves. Examples include, but are not limited to, cadaveric valve, a xenobiotic valve, or a synthetic valve. In some embodiments, the valve is a tissue valve (e.g., bio-prosthetic valve). In some embodiments, the valve is a valve for use in TAVR.

In some embodiments, the valve is a homograft or an autograft. In an autograft, a valve from the same patient (e.g., a pulmonary valve) is moved to a different position (e.g., to the aortic position) in a Ross procedure (See e.g., Yacoub M, et al., (2006). J Heart Valve Dis. 15 (4): 531-9; herein incorporated by reference in its entirety). In some embodiments, a pulmonary allograft (valve taken from a cadaver) is then used to replace the patient's own pulmonary valve. In some embodiments, when the valve is removed, and before transplanting, it is treated with an adenosine producing enzyme as described herein (e.g., in the operating room).

In some embodiments, a homograft (e.g., obtained from Cryofile, Kennesaw, Ga.) or other source is used. In some embodiments, such homografts (e.g., cryopreserved human valves from a cadaver) are used. In some embodiments, such valves are treated with adenosine producing enzymes described herein before implantation.

The present disclosure is not limited to a particular method of providing the enzyme to the valve. In some embodiments, enzymes are associated with the surface of the valve or integrated into the valve. In some embodiments, the enzymes are associated with the valve prior to implantation. In some exemplary embodiments, enzymes are crosslinked to the valve surface. The present disclosure is not limited to particular crosslinking agents. Examples include, but are not limited to, dimethyl suberimidate, the N-Hydroxysuccinimide-ester crosslinker B S3, formaldehyde, or a carbodiimide crosslinker. In some embodiments, the crosslinker is a succinimidyl-diazirine crosslinker.

In some embodiments, the enzymes are associated with the valve during or after placement of the valve in the subject. For example, in some embodiments, the enzymes are provided as a solution or are expressed by a cell (e.g., bacterial cell).

In some embodiments, enzymes are provided to a subject after implantation of the valve (e.g., in addition to enzyme already associated with the valve or instead of such enzymes), for example, via a targeting agent that targets the enzyme to the valve.

The compositions and methods described herein find use in the treatment of a variety of disorders involving heart valves. Examples include, but are not limited to, congenital heart disease, stenosis, or leaky valves (insufficiency).

The compositions and methods described herein find use in the replacement of any valve (e.g., heart valve). In some embodiments, the valve is a mitral valve (bicuspid valve), tricuspid valve, aortic valve, or pulmonary valve.

In some embodiments, the compositions and methods find use in the treatment of aortic valve disease. Aortic valve replacement is most frequently done through a median sternotomy, meaning the incision is made by cutting through the sternum. Once the patient is on bypass, a cut is made in the aorta and a crossclamp applied. The surgeon then removes the patient's diseased aortic valve and a mechanical or tissue valve is put in its place. Once the valve is in place and the aorta has been closed, the patient is taken off the heart-lung machine. Transesophageal echocardiogram (TEE, an ultra-sound of the heart done through the esophagus) can be used to verify that the new valve is functioning properly. Pacing wires are usually put in place, so that the heart can be manually paced should any complications arise after surgery. Drainage tubes are also inserted to drain fluids from the chest and pericardium following surgery. These are usually removed within 36 hours while the pacing wires are generally left in place until right before the patient is discharged from the hospital.

In some embodiments, the compositions and methods described herein find use in TAVR, the replacement of the aortic valve of the heart through the blood vessels (as opposed to valve replacement by open heart surgery). The replacement valve is delivered via one of several access methods: transfemoral (in the upper leg), transapical (through the wall of the heart), subclavian (beneath the collar bone), direct aortic (through a minimally invasive surgical incision into the aorta), and transcaval (from a temporary hole in the aorta near the belly button through a vein in the upper leg), among others.

### Experimental

The following examples are provided to demonstrate and further illustrate certain embodiments of the present disclosure and are not to be construed as limiting the scope thereof.

### EXAMPLE 1

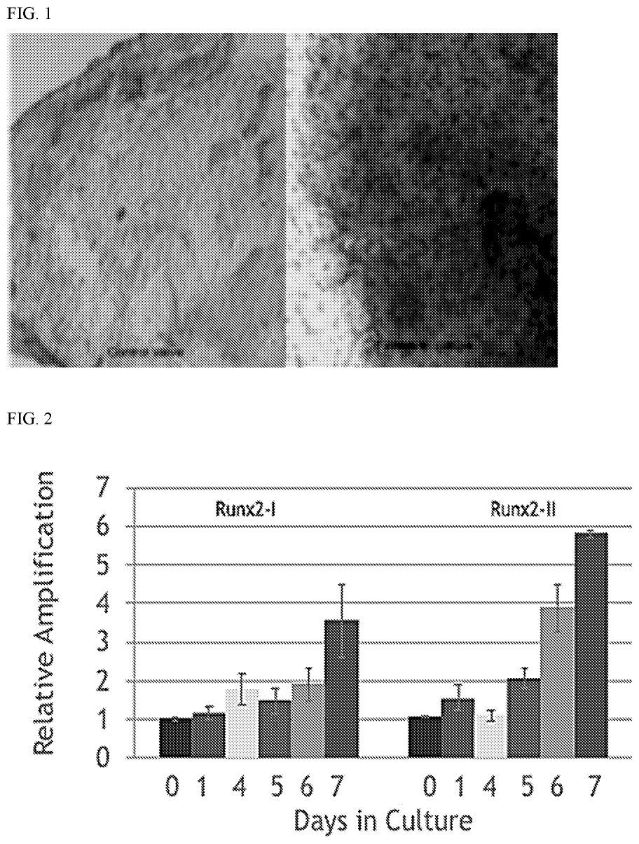

Cellularization of valves in the heart is mediated by a tissue interaction (Runyan and Markwald, 1983). Runx2 is a mesenchymal cell marker (Gitler et al., 2003). It was found that Runx2 was expressed in the embryonic heart as the Runx2-I isoform and that Runx2-I is regulated by TGFβ (Tavares et al., 2018). As Runx2 is heavily associated with calcification and osteocyte commitment, isoform expression in a whole organ model of valve calcification was investigated using avian fetal valve leaflets (Peacock et al., 2010). Atrioventricular (AV) canal valves incubated on the surface of collagen gels in conventional media develop calcifications (FIG. 1). By day 7, both Runx2 isoforms were expressed in these explants (FIG. 2).

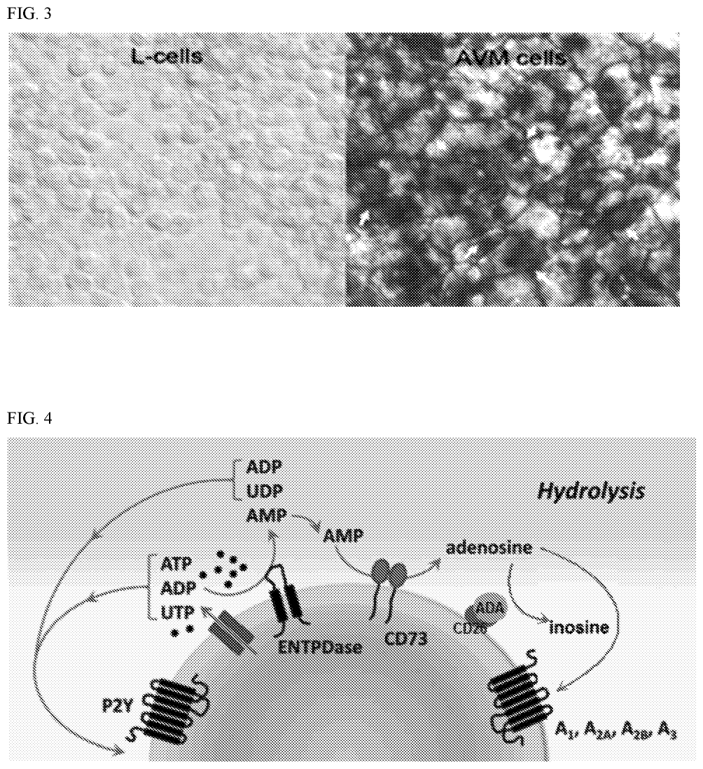

Embryonic precursors of valvular interstitial cells (VICs) calcify when grown as tissue culture aggregates similar to micromass cultures of limb cells developed by (Ahrens et al., 1977). A mouse fetal VIC cell line (AVM cells) obtained from the Immortamouse (gift of Kai Jiao, UAB) were used to investigate calcification. These cells express a temperature sensitive Large T antigen and are immortal at 33° C. and revert to a valvular phenotype at 37° C. As shown in FIG. 3, calcification was produced in vitro and both Runx2 isoforms were upregulated during the calcification process. Knockdown of Runx2-II in the mouse demonstrated that Runx2-I is capable of mediating calcification by itself (Okura et al., 2014). Additional markers of calcification expressed in these assays are shown in Table 1.

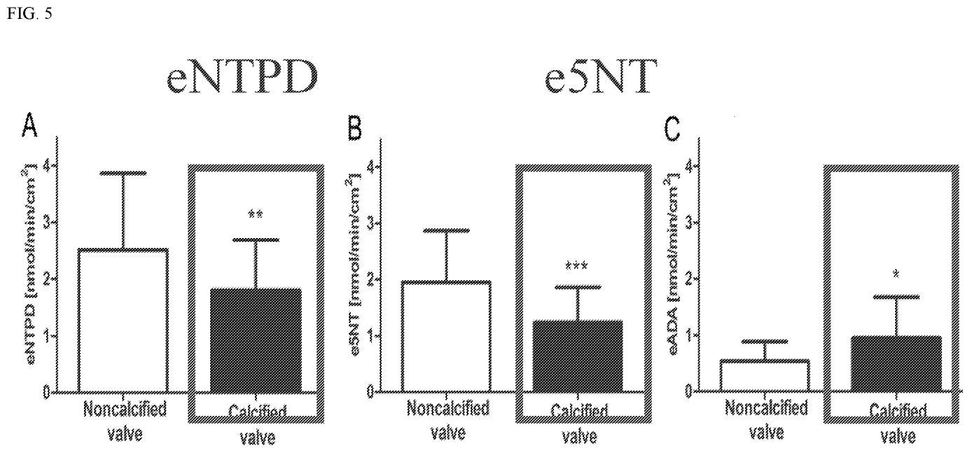

Nucleotides are present in the extracellular space and play an important role in moderating inflammation (Blackburn et al., 2009). Extracellular nucleotides act via receptors on a cell surface as signaling molecules through two major families of purine receptors, P1 for nucleosides and P2 for nucleotides. Activation of P2 receptors results in a stimulation of signal transduction pathways, which tend to oppose the signals by P1 receptors. Broadly, extracellular nucleotides stimulate inflammation while nucleosides attenuate it (Di Virgilio and Vuerich, 2015). Both adenine nucleotides and nucleosides are metabolized by cell surface ecto-enzymes (FIG. 4). Extracellular ATP is hydrolyzed to AMP by CD39 (ecto-nucleoside triphosphate diphosphohydrolase 1, eNTPDase1) and by ectonucleotide pyrophosphatase/phosphodiesterase 1 (eNPP1). Subsequently, extracellular AMP is converted to adenosine by CD73 (ecto-5′-nucleotidase, e5′NT) or alkaline phosphatase (ALP). CD73 is contemplated to be the primary enzyme capable of extracellular AMP hydrolysis. Adenosine is finally converted to inosine via ecto-adenosine deaminase (eADA). Adenosine, produced by CD73, demonstrates anti-inflammatory, antithrombotic and vasodilatory properties (Layland et al., 2014). It is a ligand for four G protein-coupled receptor subtypes—A1, A2A, A2B and A3 with A1 and A2A having the highest affinity (Headrick et al., 2013).

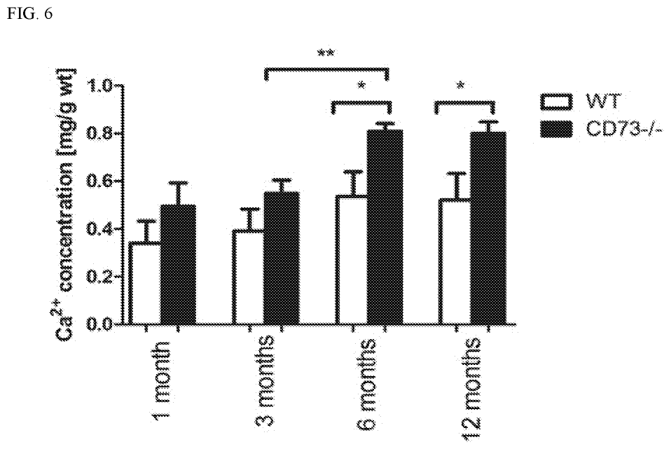

Studies on the inflammatory component of calcific valve disease and adenosine and its production by ecto-nucleotidases on the surfaces of cells mediating valve pathology are described in Khalpey et al. (Khalpey et al., 2005). These studies demonstrated a decrease in CD73 activity in pig hearts following exposure to human blood. This impairs adenosine production and contributed to xenograft rejection. A recent study (Kutryb-Zajac et al., 2018) investigated extracellular nucleotide metabolism and the content and function of resident immune cells in human calcific aortic valve. It was found that activities of enzymes engaged in ATP and AMP hydrolysis were inversely correlated with the concentration of calcification in the aortic valve (FIG. 6).

Experiments also showed that valvular endothelial cells and VICs expressed predominantly CD39 and CD73, which are responsible for the hydrolysis of ATP and AMP respectively. Immune cells derived from a calcific valve represented a poor source of CD73. All populations of immune cells expressed a significant activity of adenosine deaminase. The major observation was that calcified valves showed diminished activities of nucleotide-hydrolyzing ectoenzymes concomitant with an increased activity of ecto-adenosine deaminase. Inflammatory infiltrate cells add additional ecto-adenosine deaminase to the aortic valve, which causes adenosine degradation. Reduction in the total activity of ATP and AMP hydrolyzing enzymes (CD39, CD73) with a simultaneous increase in adenosine deamination rate would lower the adenosine level to favor valve inflammation and calcification (FIG. 6).

In a recently published study (Zukowska et al., 2017), C57BL/6J with CD73 knockout (CD73−/−) mice, treated with normal-fat and high-fat diet. The AMP hydrolysis rate on the aortic valve surface in the CD73−/− mice was approximately ten times lower in comparison with the WT. Aortic valve area was significantly smaller in the CD73−/− regardless of the diet treatment, compared to WT. The mineralization level of the aortic valves was significantly higher in the aortic valve leaflets of CD73−/− mice compared to WT. Alkaline phosphatase (ALP) activity was increased after ATP treatment and reduced after adenosine treatment in aortas incubated in osteogenic medium. This study provides experimental evidence that CD73 deletion leads to development of the aortic valve dysfunction in mice as evidenced by the structural and functional changes.

Mahmut et al. (Mahmut et al., 2015) found that adenosine A2A receptors increase expression in calcified human aortic valves as adenosine A1 receptors disappear. This data is inconsistent with results from recent work from the Smolenski lab (Kutryb-Zajac et al., 2018) showing no changes in adenosine receptor expression between healthy and calcified aortic valves. Further evidence demonstrated that specific receptors such as P2Y2, P2Y4, P2Y6, P2X3, or A2B contribute to increased expression of Runx-2 and other osteogenic markers (Costessi et al., 2005). Purinergic receptors have a widespread distribution on many human cells. Four subtypes of adenosine receptors, seven subtypes of P2X receptors and eight subtypes of P2Y receptors enable a complex response to extracellular nucleotides and nucleosides. Purinergic signaling is involved in embryonic and stem cell development (Burnstock and Ulrich, 2011) and also in physiology and pathophysiology of the nervous system (Burnstock, 2011). Purinergic receptors are a current pharmacological target for treatment of thrombosis, heart failure, renal failure, hypertension, atherosclerosis, migraine, pain, Parkinson's disease, schizophrenia, inflammation and inflammatory disorders, colon carcinoma, asthma, arrhythmia, myocardial and brain ischemia, diabetes, and obesity (Burnstock, 2006; Jacobson and Gao, 2006). A number of pharmaceuticals, targeting purinergic receptors, are in clinical testing.

The cell and organ culture models described above are used to study valve calcification by staining and expression of osteogenic markers. The CD73 null mouse model shows valvular calcification consistent with a loss of extracellular adenosine. Both Runx2 isoform levels are elevated concomitant with calcification in vitro and in situ. To explore the relationship between identified regulators and markers of the osteogenic program in calcifying valve tissues, Runx2 isoform and osteogenic marker expression was measured in calcifying AVM cell cultures with various treatments. AVM cultures were treated with siRNA against Runx2-II, noggin (inhibitor of BMP), an anti-TGFβ antibody (against all three isoforms), and DAPT (inhibitor of Notch signaling). Runx2 siRNA inhibited expression of both Runx2 isoforms as well as transcription factors Twist1 and Cbfβ and the calcification marker, MGP. Inhibition of TGFβ and BMP signaling had little effect on Runx2 expression and lesser effects on the other markers than loss of Runx2. Inhibition of Notch reduced Runx2 expression but upregulated the other markers. These indicate that Runx2 is a fairly central player in this calcification process but that Runx2 expression, in these cultures, is independent of TGFβ and BMP signaling.

Twist1 gene dosage plays an important role in bone formation through regulation of Runx2. Twist1 is a negative regulator of Runx2 by binding to the Runx DNA-binding domain and inactivating its induction of gene expression (Bialek et al., 2004). Runx2^{+/-} mice have delayed osteoblast differentiation and closure of skull bones. Mice heterozygous for both Runx2^{+/-} and Twist1^{+/-} have a normal skull and bone development (Bialek et al., 2004). These results and other studies indicate genetic interaction between Twist1 and Runx2. Twist1 is known to be expressed in calcifying valves (Chakraborty et al., 2010). As Twist1 and Runx2 are common to both EMT and valve calcification, the regulation of this interaction may be significant.

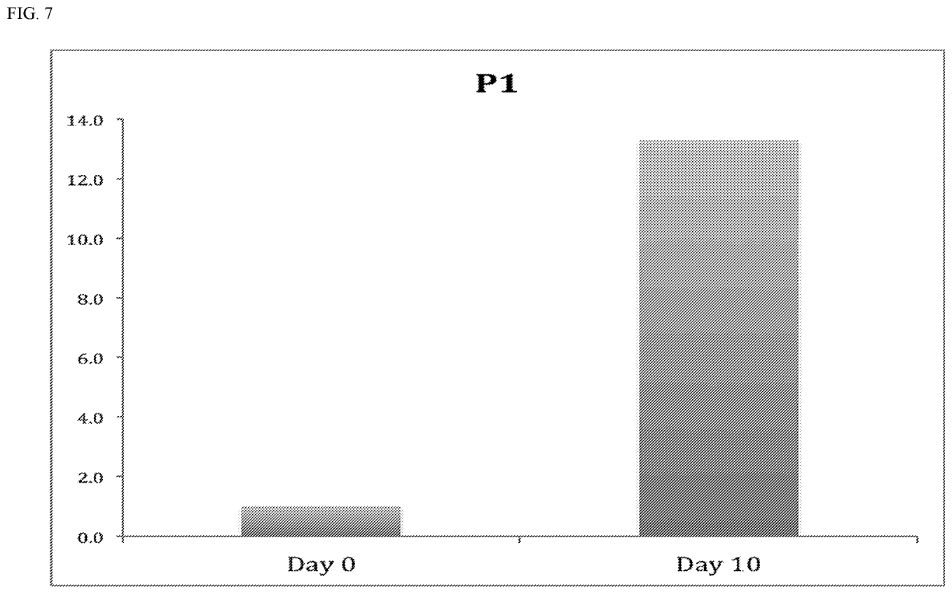

Runx2 proteins also interact with a cofactor, Cbfβ or core binding factor beta (Kanatani et al., 2006). Cbfβ binds to the DNA binding region of Runx2 and appears to regulate the ability of Runx2 to bind to DNA (Kundu et al., 2002). There is a report that Cbfβ binds with greater affinity to Runx2-I than Runx2-II but the significance of this observation is poorly understood (Kanatani et al., 2006). There is also evidence to indicate that Cbfβ stabilizes Runx2 proteins (Qin et al., 2015). There are two isoforms of Cbfβ produced by alternative splicing and while redundant in bone, they vary in potency in regulating Runx2 activity (Jiang et al., 2016). Culture of fetal chick valves and mouse AVM cells established that is it possible to observe calcification ion culture, even in the absence of specific osteogenic media. However, both tissues were of developmental origin and from the atrioventricular canal. An aortic valve leaflet was obtained from a subject during surgery for replacement of valvular insufficiency. The valve was divided into segments and one segment was cultured for 10 days in organ culture with a conventional valve medium (DMEM with ITS supplement and 1% FBS). The other segment was placed in RNAlater. Both segments were extracted for RNA and Runx2-II and MGP mRNAs were measured. In this patient's tissues, incubation in the calcification protocol for 10 days increased Runx2-II expression 13.3 fold (FIG. 7) and MGP 2.2 fold.

The data show significant induction of osteogenic markers (Runx2 and MGP) after mouse VIC incubation with agonists of A1 (MeCCP) and A2A (PSB0777) adenosine receptors. Published data (Mahmut et al., 2015) indicated that the A1 receptor inhibits calcification in cell cultures while the A2A receptor enhanced consistent with work of (D'Alimonte et al., 2013) which indicated that the stimulation of A1 receptor favors the commitment of MSCs towards osteogenesis. These data support a role for an adenosine signaling pathway in the regulation of calcification.

### EXAMPLE 2

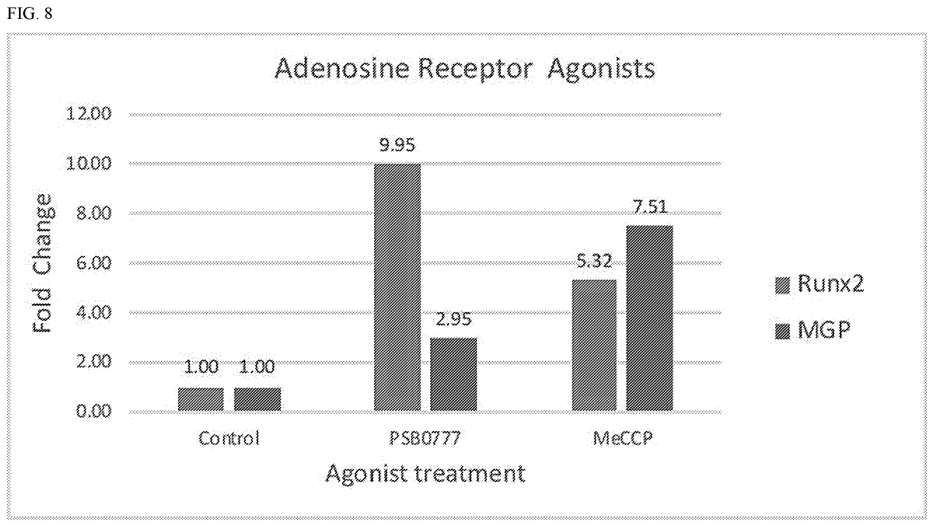

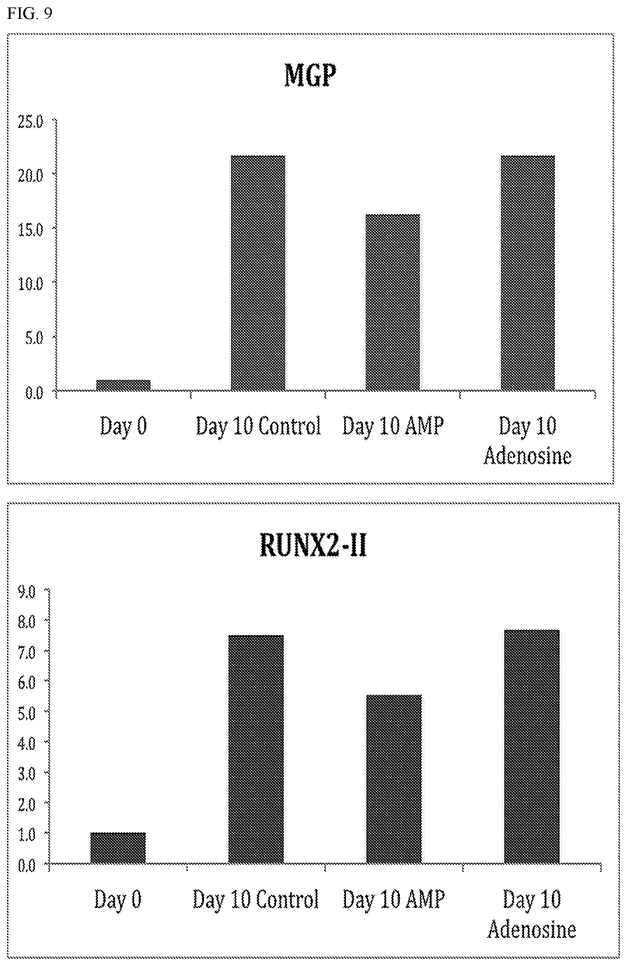

This example describes identification of marker progression in vitro. In vitro analysis begins with organ culture of porcine aortic valve segments, human aortic valvular interstitial and endothelial cells and mouse AVM cells. It is confirmed that the cell lines replicate the findings of (Kaniewska-Bednarczuk et al., 2018) and are consistent between species. For organ culture, the approach of Peacock et al (2010) is used except that porcine aortic valve leaflets are cultured on the surface of collagen gels as in FIG. 1. This approach has been successful for human aortic valves as well (FIG. 8) but human valves collected at the time of replacement would likely have varied levels of existing calcification. Valves obtained from young pigs would have little calcification. Data obtained from these cell and organ cultures is collected for an aggregated response but the sex of the source is identified and data evaluated for potential differential response by sex.

For cell culture, human and porcine VICs and human valvular endothelial cells (VECs) are used. VICs are expanded from initial culture and stored as aliquots in liquid nitrogen. All experiments are performed with low passage number cells and each cell source is profiled by the UA Genetics Core to confirm and maintain the identity of each cell line. A mouse AV valve mesenchymal cell line (AVM) was obtained from Kai Jao (U. of Ala., Birmingham). These cells were from mice containing a temperature sensitive large T transgene (Immorta-Mouse) and the cells were grown at 33° C. to obtain an immortal cell line (Peng et al., 2017). The cells revert to a cushion/valve phenotype at 37° C. The micromass cultures of Solursh and colleagues (Ahrens et al.,1977) were recapitulated to observe calcification in vitro in this cell system. Calcium deposits became visible by von Kossa staining by 5 days in culture and prominent by day 12 (FIG. 3). The response shows specificity by cell type as a fibroblast cell line grown under identical conditions shows no sign of calcification. Marker expression is consistent with a valve calcification phenotype and supports a central role for Runx2 in calcification (FIG. 2). For organ culture of intact valves, fresh aortic pig valves are obtained from the meat lab in the College of Agriculture and incubated on the surface of collagen gels. Experiments compare responses in intact aortic valves and VIC and VEC cell lines. The experimentation reveals any differences between valve types. Though 19 purinergic receptors are identified in Table 1, those P2Y and P2X receptors that are not found in valve tissues are excluded from further analysis.

TABLE 1
Markers for Evaluation of Calcification
and Ecto-nucleotidase activity

| Transcrip- | | Ecto- | |
|---|---|---|---|
| tion | Osteogenic | nucleotidase | |
| Factors | Markers | pathway | Purinergic Receptors |
| Runx2-I | MGP | CD39 | P2Y1, P2Y2, P2Y4, P2Y6, |
| | | | P2Y11, P2Y12, P2Y13, P2Y14 |
| Runx2-II | OP | CD73 | P2X1-7 |
| Cbfb1 | | ALP | A1 AR, A2A AR, A2B AR |
| Cbfb2 | | eNPP1 | |
| Twist1 | | | |
| Sox9 | | | |

Experiments examine the time course of marker expression during calcification in vitro. Organ and cell cultures are prepared and collected daily for RNA collection until day 12. qPCR is performed for markers in Table 1. The progress of calcification is monitored by staining cultures with alizarin red on culture days 3, 6, 9 and 12. Additional day 12 cultures are collected for Western Blots to examine concordance between measures of transcription and translation. These experiments identify each cell type with the progression of marker gene expression during calcification in vitro. Organ cultures show the overall pattern of progression in a mixed tissue.

### EXAMPLE 3

This example describes identification altered marker expression after knockdown of purinergic receptors and ectoenzymes. To explore marker responses among calcifying cultures related to purinergic signaling, cultures are prepared as above and treated with siRNAs targeting purinergic receptors identified in valve tissues (A1AR, A2aAR, A2bAR, and select identified P2X and P2Y receptors). Though initial experiments in the first experiment examine human VICs, porcine VICs, human VECs, mouse AVM and porcine mitral valves, subsequent experiments are focused on a few cell and tissue types based upon those that are consistent and informative relevant to human tissues. qPCR is performed for markers in Table 1 to establish altered regulation in response to specific receptors. It is important to characterize any co-regulation between the various osteogenic markers to the ecto-nucleotidases and specific purinergic receptors.

To simulate pathobiology of aortic valve tissue, inhibition of ectoenzymes are performed to avoid rapid degradation of ATP through ADP→AMP→adenosine to inosine. In order to evaluate this, specific ecto-enzymes are inhibited in human VICs colonies with concomitant measurement of osteogenic markers. In one experiment CD73, adenosine deaminase and ALP are inhibited to keep a pool of extracellular nucleotides intact that would bind to P2X and P2Y receptors. This experiment simulates CD73 deficiency, which is a rare condition called Arterial Calcification Due to Deficiency of CD73 (ACDC) (Rutsch et al., 2011). In the second experiment, CD73 is preserved and adenosine deaminase and ALP are silenced. This converts all of nucleotides to adenosine and conversion of adenosine to inosine is blocked. This step stimulates all expressed adenosine receptors and lowers stimulation by P2X and P2Y receptors. In a subsequent experiment, added adenosine deaminase shifts all nucleotides and adenosine to inosine. In this condition, stimulation of purinergic receptors is poor, and this condition mimics inflammation as a lymphocytes are potent source of adenosine deaminase (Gorrell et al., 2001). In all these experiments, content of nucleotides and nucleosides are systematically evaluated by HPLC.

### EXAMPLE 4

This example describes in vitro assessment of adenosine and other purinergic receptor agonists and antagonists. In vitro cultured human aortic valves, human aortic VICs, and mouse AVM cells are cultured with agonists and antagonists and evaluated for marker expression by qPCR and western blot. Additional cultures are treated with forskolin as an activator of adenyl cyclase. Short term cultures (4-6 hr) are extracted and examined for immediate early regulation of Runx2 isoforms and other osteogenic transcription factors (Table 1, Column 1). Ten to 12 day cultures are examined for marker expression under protocols that compare daily replacement of treatment to replacement every 3 days to assess the potential for sensitization. A variety of receptor specific agonists and antagonists are available. Where results are significant and alternatives are available, data for agonists or antagonists with similar receptor specificity are compared.

TABLE 2
Adenosine Receptors and Reagents

| Receptor | Agonist | Source | Antagonist | Source |
|---|---|---|---|---|
| A₁ | CCPA, | Sigma C7938 | DPCPX | Sigma |
| | 5′C15′d-(±)- | Sigma E111 | BG9719 | 119147 |
| | ENBA | | | |
| | 2′-MeCCPA | Tocris | 1,3-Dipropyl-8- | MedKoo |
| | GR79236 | Sigma G5794 | phenylxanthine | Tocris |
| | | | Rolofylline | Sigma |
| | | | (KW 3902) | K3769 |
| A_{2A} | PSB 0777 | Tocris | SCH 58261 | Sigma |
| | ATL-146e | Sigma 119137 | SCH 442416 | S4568 |
| | CGS21680 | Tocris | Preladenant | Tocris |
| | | | (SCH 420814) | |
| | Binodenoson | AX Molecules | Istradefylline | MedKoo |
| | (WRC-0470) | Inc | (KW 6002) | Tocris |
| | | | SYN 115 | AdooQ |
| A_{2B} | BAY 60-6583 | Tocris | ATL802 | MedKoo |
| | | | ISAM 140 | Tocris |
| | | | GS-6201 | Tocris |
| | | | (CVT-6883) | |
| A₃ | Cl-IB-MECA | Sigma C277 | MRE 3008F20 | Tocris |
| | MRS 5698 | Tocris | | |

### EXAMPLE 5

This example describes AVM and hVIC cells treated with soluble ecto-nucleotidases in vitro. Cultures of AVM cells and hVICs are placed in micromass culture to stimulate calcification as seen in FIG. 4. The effects of exogenous CD73 and the combination of exogenous CD39 and CD73 on the progression of transcription factors, osteogenic markers and endogenous expression of ecto-nucleotidase pathway components (Table 1) is evaluated by qPCR and western blotting. Variables in the analysis include the concentration of enzymes added to the cultures as well as the concentrations of ATP and AMP endogenous to the cultures or exogenously added to the cultures. Measurement of nucleotides (Smolenski et al., 1991) and enzyme activities are performed as published (Kaniewska-Bednarczuk et al., 2018). The objective is to identify whether the addition of CD73 or perhaps CD73 and CD39 together inhibit the calcification process in vitro. It is also explored whether addition of β-glycerophosphate or lysophosphatidylcholine in osteogenic media alters the enzymatic activity of endogenous or exogenous ecto-nucleotidases (Wiltz et al., 2014) Additional experiments with erythro-9-(2-hydroxy-3-nonyl)adenine hydrochloride (EHNA), an inhibitor of adenosine deaminase, explore the effect on calcification when the adenosine to inosine pathway is inhibited (FIG. 5).

### EXAMPLE 6

This Example investigates the influence of modified bioprosthetic valve surfaces with CD73 and CD39 on inflammatory infiltration and osteogenic markers. One extension of the role of ecto-enzymes in valve calcification is to test whether modification of the valve to manipulate the local delivery of adenosine alter the rate of calcification in vitro. To investigate this, recombinant CD73 or a combination of CD73 and CD39 is coupled to prosthetic valve surfaces. Modified valve leaflets are tested for loss or reduction of calcification markers by human VICs, VECs and AVM cells when grown on the leaflet substrates or used in a bioreactor. Bioreactor studies seed VECs and whole blood into the flow to provide a combination of endothelial and inflammatory cells.

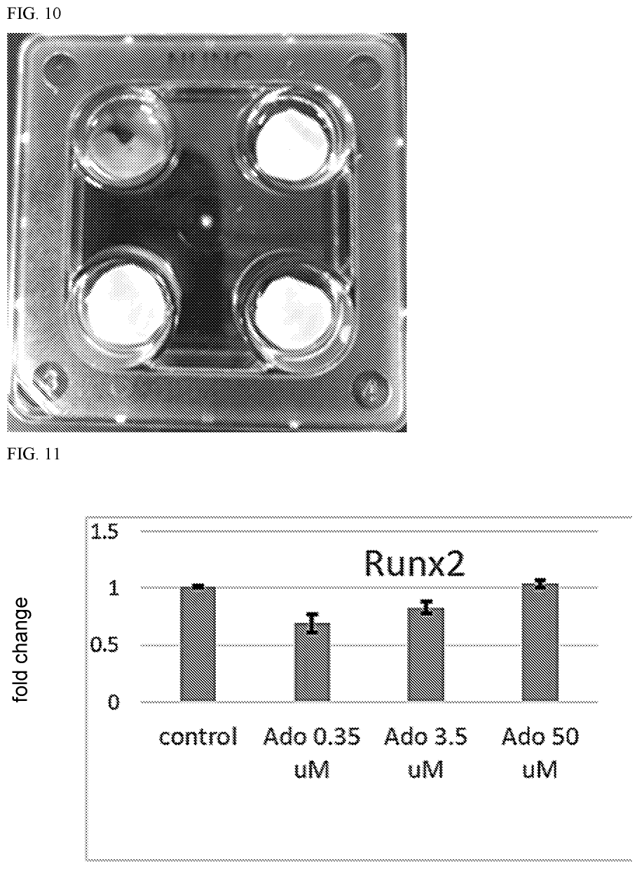

A supply of expired bovine pericardial bioprosthetic valves was obtained from a manufacturer (Edwards Model 3300TFX) to use as substrates. Valve leaflets are removed from the valves and rinsed extensively with sterile saline to remove the glutaraldehyde storage solution. Recombinant CD73 and CD39 (Sigma Aldrich) are each incubated with the heterobifunctional crosslinker, Sulfo-LC-SDA (Thermo Fisher). This succinimidyl-diazirine crosslinker is first incubated with each enzyme and then the enzyme mixture is applied to the pericardial membrane substrate and coupled via UV photoactivation to the valve surface. Valve substrates are prepared with both CD73 and a combination of CD73 and CD39. The two step coupling procedure allows assays of enzyme activity both after the initial incubation with the crosslinker and after photocoupling to the substrate. Glutaraldehyde from bioprosthesis is quenched by the incubation in 0.1 M NaBH4 (Kim et al., 2011). Enzyme concentrations in the coupling solution is varied to produce a range of enzyme densities. Confirmation that the enzyme is coupled to the substrate and that it is active is demonstrated by a staining for release of phosphate from the nucleotide substrate (Wachstein and Meisel, 1957) (FIG. 10).

In vitro experiments the valve leaflets as culture substrates with humanVlCs and mouse AVM cells in high density culture. Cells are grown on the substrates for 10 days with unmodified valve leaflets and CD73, CD39 or CD39/CD73 modifications. Cultures are extracted for RNA and qPCR is used to evaluate the extent that osteogenic markers are reduced by the modified substrates.

### EXAMPLE 7

This Example describes the effects of substrate modification on bioprosthetic colonization. Bioprostheses with coupled CD73 protein with or without CD39 protein is inserted into a bioreactor (Converse et al., 2017) circulating whole human blood and incubated up to 10 days. The bioreactor is one described by (Sierad, 2009). Some experiments include delivery of VEC cells in combination with whole blood. Control substrates are evaluated for the stability of enzyme coupling and activity over time in the presence or absence of whole blood by collection and assay at intervals. To ensure stability of the incubation environment, blood is replaced every day and controlled for blood cells, ions, hematocrit and microbial contamination. In order to induce a wide spectrum of leukocytes, proinflammatory cytokines (IL-2, IL-6 and TNF-α) (Badylak et al., 2008) are added. On completion, the bioprosthesis is removed from the bioreactor and high-resolution photos are taken to evaluate the thrombus. Analysis includes immunohistochemistry to show localization of CD73 and CD39 and qPCR of cells extracted from one leaflet for osteogenic markers. The remaining two leaflets are treated with trypsin and collagenase to release attached cells for analysis. Identification of the attached cell types is performed by use of single cell RNAseq. With this technique, one can identify the subsets of cell types attached to the bioprosthesis from whole blood and gene expression patterns altered by the substrate modifications. The 10x Genomics Chrome platform is used to produce a scRNAseq library and evaluate exon expression by cell types in the inflammatory infiltration. This platform uses a microfluidic chip approach to bar code the cDNA generated from each cell for up to 80,000 cells in a mixed preparation. Infiltrated bioprosthesis are dissected, and enzymatically dissociated to single cells. Single cell preparations are sorted for viability by FACS with Sytox Green. Approximately 1,700 cells are loaded into each channel of a 10X Genomics Chromium System for each valve sample. Samples are coded and processed blindly and only identified after data collection is complete. Following capture and lysis, cDNA is synthesized and amplified for 12 cycles per the manufacturer's protocol. The resulting amplified cDNA libraries are used for high throughput sequencing on an Illumina NextSeq 500. Raw data is evaluated, trimmed and aligned to the human transcriptome and deconvoluted using Cell Ranger software (10X Genomics) to assign cell identity based upon the most highly expressed genes for each cell. As cell identity determination is compromised by “dropouts” where transcripts are missed by the limited number of transcripts per cell and the limited number of reads/cell, the data is normalized by scImpute to improve cell assignments. The data is displayed by t-SNE. Such data has proven useful for the characterization of inflammatory infiltration as it can distinguish the multiple cell types present in a tissue and the subsets of each cell type that show the range of gene expression. The data identifies both optimal markers for each cell type and any subtypes in the sample based upon the 500-1000 most highly expressed genes.

### EXAMPLE 8

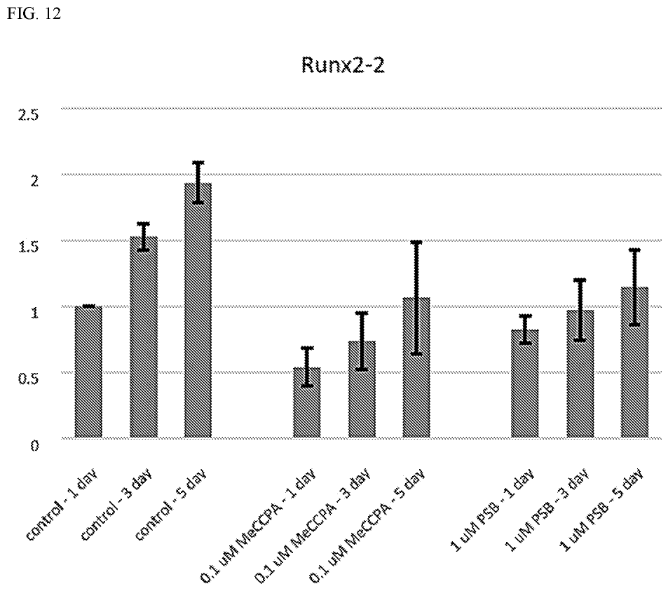

This example describes the effects of adenosine and adenosine receptor agonists on Runx2 and osteogenic markers. To test the direct role of adenosine in calcification, AVM cultures were maintained at 0.35 μM, 3.5 μM and 50 μM concentrations of adenosine to reflect the relative affinities of the A1, A2a and A2b adenosine receptors. Adenosine levels were confirmed using HPLC. As shown in FIG. 11, Runx2 expression was inhibited at an exposure consistent with the high affinity A1 receptor. Treatment with agonists towards both the A1 (0.1 μM 2′-MeCCPA) and A2a (1 μM PSB0777) adenosine receptors reduced the expression of Runx2 compared to the progression of control cultures (FIG. 12).

### EXAMPLE 9

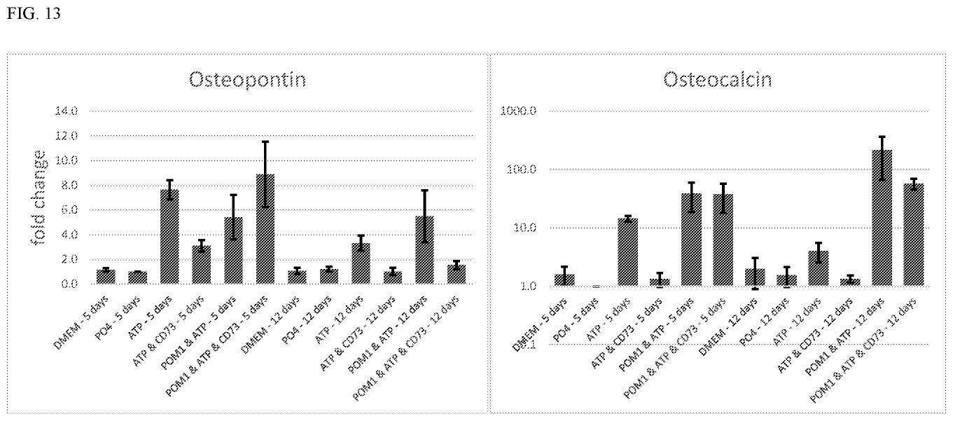

This example describes data demonstrating that ATP is procalcific in valve cell cultures. Experiments show that addition of PO4 to culture media increases osteogenic markers at 12 days (FIG. 13). At 5 days, before osteogenic markers are overtly upregulated in controls, exogenous 100 μM ATP (added every 12 hr) produces a large increase in osteocalcin and osteopontin. Over 5 days, exposure to this concentration of ATP is apoptotic as measured by the ratio of Bax/Bcl2, and this may contribute to calcification. These effects are reduced by addition of exogenous CD73 enzyme to enhance the conversion of ATP to adenosine.

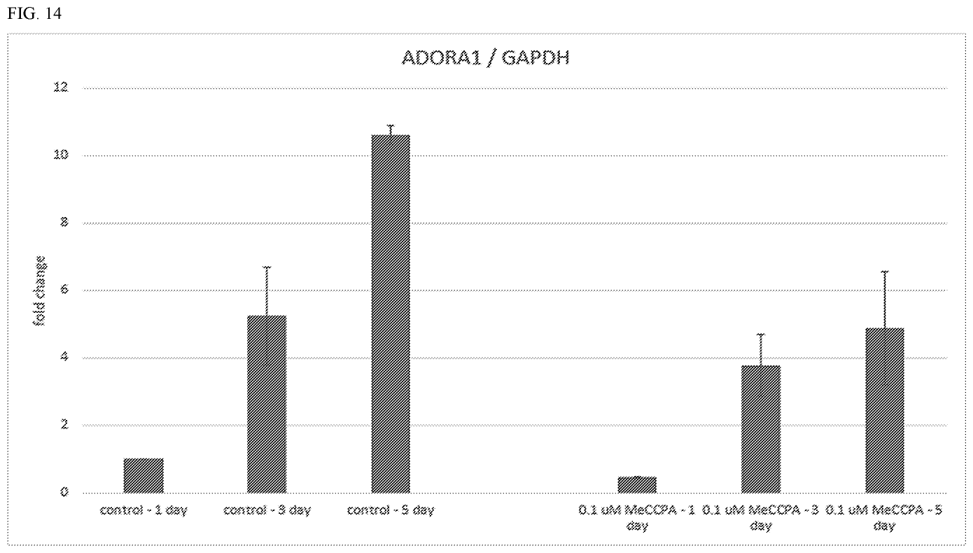

FIG. 14 shows that the addition of 1 μg/ml of recombinant CD73 to micromass cultures (approximately 100,000 cells in a dish) increases the concentration of adenosine in the culture media from 1.7 to 8.7 M (5-fold). This change in concentration is sufficient to activate the A1 and A2a but not the A2b receptor (EC50=0.3, 0.7 and 23.5 μM, respectively).

All publications, patents, patent applications and accession numbers mentioned in the above specification are herein incorporated by reference in their entirety. Although the invention has been described in connection with specific embodiments, it should be understood that the invention as claimed should not be unduly limited to such specific embodiments. Indeed, various modifications and variations of the described compositions and methods of the invention will be apparent to those of ordinary skill in the art and are intended to be within the scope of the following claims.

1. An implantable medical valve comprising one or more enzymes that generate adenosine.

2. The valve of claim 1, wherein said enzyme is an ecto-nucleotidase or an ecto-nucleosidase.

3. The valve of claim 1, wherein said ecto-nucleotidase is selected from the group consisting of a 5′ ectonucleotidase, an ecto-nucleoside triphosphate diphosphohydrolase 1, a nucleotide pyrophosphatase/phosphodiesterase (NPP)-type ecto-phosphodiesterase, and an alkaline phosphatase.

4. The valve of claim 3, wherein said 5′ ectonucleotidase is CD73.

5. The valve of claim 3, wherein said ecto-nucleoside triphosphate diphosphohydrolase 1 is CD39.

6. The valve of claim 1, wherein said enzymes are a combination of CD73 and CD39.

7. The valve of claim 1, wherein said valve is a heart valve.

8. The valve of claim 7, wherein said heart valve is selected from the group consisting of a cadaveric valve, homograft, autograft, a xenobiotic valve, and a synthetic valve.

9. The valve of claim 7, wherein said valve is a valve for use in Transcatheter Aortic Valve Replacement (TAVR).

10. The valve of claim 1, wherein said enzyme is present on the surface of said valve or are integrated into said valve.

11. The valve of claim 10, wherein said enzyme is cross-linked to said valve.

12. The valve of claim 10, wherein said enzyme is secreted by a cell that expresses said enzyme.

13. The valve of claim 1, wherein said enzyme is modified to prevent proteolysis of said enzyme.

14. The valve of claim 13, wherein said modification is selected from the group consisting of removal of proteolytic target sites and truncation of said enzyme.

15. (canceled)

16. The valve of claim 1, wherein said enzyme is linked to a targeting agent that binds to said medical valve.

17. The valve of claim 16, wherein said targeting agent is an antibody.

18. The valve of claim 16, wherein said valve comprises a binding target for said targeting agent.

19. The valve of claim 18, wherein said binding target is a hapten.

20. A system, comprising:
a) an implantable medical valve; and
b) a source of an enzyme that generates adenosine.

21-39. (canceled)

40. A method of preventing calcification of an implantable medical valve, comprising:
providing the valve or system of claim 1 to a subject in need thereof.

41-43. (canceled)
